(19) United States
(12) Patent Application Publication
(10) Pub. No.: US 2015/0209364 A1
(43) Pub. Date: Jul. 30, 2015

(54) METHOD FOR TREATING EPIDERMOID CARCINOMA

(71) Applicant: Johnpro Biotech Inc., New Taipei City (TW)

(72) Inventors: Yu-Shan Wang, New Taipei City (TW); Yi-Chun Huang, New Taipei City (TW); Chao-Chun Chang, New Taipei City (TW); Kwan-Hwa Chi, New Taipei City (TW); Pei-Ru Liao, New Taipei City (TW)

(21) Appl. No.: 14/677,271

(22) Filed: Apr. 2, 2015

Related U.S. Application Data
(63) Continuation-in-part of application No. 13/238,649, filed on Sep. 21, 2011.

(30) Foreign Application Priority Data
Feb. 23, 2011 (TW) 100106076

(51) Int. Cl. A61K 31/5377; A61K 31/4706; A61K 31/436

(52) U.S. Cl. CPC A61K 31/5377; A61K 31/436; A61K 31/4706

(57) ABSTRACT

Disclosed herein is a method for enhancing the susceptibility of a subject having epidermoid carcinoma toward a tyrosine kinase inhibitor. The method includes administering to the subject an effective amount of a targeted therapy sensitizer and an effective amount of gefitinib. According to various embodiments of the present disclosure, the targeted therapy sensitizer consists of rapamycin and a substituted quinoline, such as chloroquine. Also included herein is a pharmaceutical composition that includes an effective amount of gefitinib, an effective amount of a targeted therapy sensitizer for synergistically improving or enhancing the efficacy of the gefitinib for treating epidermoid carcinoma in a subject in need thereof, and a pharmaceutically acceptable excipient.

### CROSS-REFERENCE TO RELATED APPLICATION

This application is a continuation-in-part of application Ser. No. 13/238,649, filed Sep. 21, 2011, which claims the benefit of Taiwan Patent Application No. 100106076, filed Feb. 23, 2011, the entire contents of which are hereby incorporated by reference.

### BACKGROUND OF THE INVENTION

1. Field of the Invention

The present disclosure relates to field of treating epidermoid carcinoma; in particular, for enhancing the susceptibility of a subject having epidermoid carcinoma toward a tyrosine kinase inhibitor.

2. Description of Related Art

Lung cancer is the most common cause of death from cancer worldwide, accounting for about 1.59 million deaths in 2012. The overall ratio of mortality to incidence of lung cancer is 0.87, and because of this high fatality, targeted therapy in combination with other treatments has been considered a promising therapeutic regimen to combat lung cancer, such as non-small cell lung cancer (NSCLC).

Epidermal growth factor receptor (EGFR) is important to cancer cell growth, proliferation, invasion, and metastasis, and is frequently deregulated in NSCLCs, thereby making it a target molecule for the development of targeted therapy. Gefitinib and erlotinib are both tyrosine kinase inhibitors (TKI) capable of inhibiting the tyrosine kinase activity of EGFR, and have been developed for the treatment of NSCLC. Nevertheless, some patients do not respond to EGFR-targeted therapy, while those respond initially usually develop resistance in less than 12 months.

In many cases, resistance to EGFR-targeted therapy is associated with the activation of phosphatidylinositol 3-kinase (PI3K)/AKT/mammalian target of rapamycin (mTOR). This deregulation of PI3K/AKT/mTOR pathway in human cancer cell lead to, among others, extended cancer cell survival, enhanced cancer cell proliferation, and/or angiogenesis, thereby promoting tumor progression and metastasis.

Another common manifestation of EGFR-targeted therapy resistance is the increase in macroautophagy (or autophagy). Autophagy is a cytoprotective mechanism for cancer cells to survive under unfavorable conditions, such as nutrient- and oxygen-deficient environment.

In view of the foregoing, there exists a need in the art for providing a novel strategy for combating various mechanisms of resistance to EGFR-targeted therapy.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

In one aspect, the present disclosure is directed to a pharmaceutical composition for treating epidermoid carcinoma in a subject in need thereof. In particular, the pharmaceutical composition is used in a targeted cancer therapy, such as EGFR-targeted therapy.

According to one embodiment of the present disclosure, the pharmaceutical composition comprises an effective amount of gefitinib, an effective amount of a targeted therapy sensitizer and a pharmaceutically acceptable excipient. The targeted therapy sensitizer comprises rapamycin and a substituted quinoline, which is used for synergistically improving or enhancing the efficacy of the gefitinib for treating epidermoid carcinoma in the subject.

In certain embodiments, the substituted quinoline is chloroquine or a pharmaceutically acceptable salt thereof.

According to some embodiments of the present disclosure, the rapamycin and the substituted quinolone are present in the targeted therapy sensitizer in a weight ratio between 10:1 and 1:5,000; preferably, between 5:1 and 1:4,000; more preferably, between 2:1 and 1:3,000.

According to various embodiments of the present disclosure, the gefitinib and the targeted therapy sensitizer are present in the pharmaceutical composition in a ratio about 10:1 to 1:100 by weight; preferably, about 5:1 to 1:50 by weight; more preferably, about 1:1 to 1:25 by weight.

In certain embodiments, the epidermoid carcinoma is non-small cell lung cancer. In some cases, the non-small cell lung cancer is resistant to at least one tyrosine kinase inhibitor. Examples of tyrosine kinase inhibitors include, but are not limited to, gefitinib and erlotinib.

In another aspect, the present disclosure is directed to a method for enhancing the susceptibility of a subject having epidermoid carcinoma toward a tyrosine kinase inhibitor.

According to embodiments of the present disclosure, the method comprises the steps of, administering to the subject an effective amount of a targeted therapy sensitizer consisting of rapamycin and a substituted quinoline; and administering to the subject an effective amount of gefitinib.

In various embodiments, the drugs to be administered can be any of those described above in connection with the first aspect (and embodiments thereof) of the present disclosure.

In certain embodiments, the epidermoid carcinoma is non-small cell lung cancer. In some cases, the non-small cell lung cancer is resistant to at least one tyrosine kinase inhibitor. Examples of tyrosine kinase inhibitors include, but are not limited to, gefitinib and erlotinib.

According to various embodiments of the present disclosure, the targeted therapy sensitizer is administered prior to, concurrently with, or after the administration of gefitinib.

In some embodiments, the targeted therapy sensitizer is administered in a regimen that us the same as or different from that of gefitinib.

According to certain embodiments of the present disclosure, during a course of treatment, at least two doses of the targeted therapy sensitizer are administered to the subject; and one or more doses of gefitinib are administered between the two consecutive doses of the targeted therapy sensitizer.

In certain embodiments, the substituted quinoline is chloroquine phosphate, and the weight ratio between rapamycin and chloroquine phosphate and is 1:1 to 1:2.

In some embodiments, the subject is a human being.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1 is a histogram illustrating the percentage of apoptotic A549 cells according to Example 1 of the present disclosure;

FIG. 2A is a histogram illustrating the percentage of apoptotic H1975 cells t according to Example 2 of the present disclosure;

FIG. 2B is a photograph of the electrophoresis result according to Example 2 of the present disclosure; and

FIG. 3 provides line graphs illustrating cell viability of H1975 cells according to Example 3 of the present disclosure.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques. Ranges can be expressed herein as from one endpoint to another endpoint or between two endpoints. All ranges disclosed herein are inclusive of the endpoints, unless specified otherwise.

As used herein, the term “targeted therapy” refers to the use of one or more therapeutic agents which aims at one or more particular target molecules (e.g., proteins) involved in the tumor genesis, tumor progression, tumor metastasis, or tumor cell proliferation or repair. One target of particular interest of the present disclosure is the EGFR receptor that plays an important role in angiogenesis, and accordingly, various EGFR inhibitors, including tyrosine kinase inhibitors against EGFR (hereinafter, EGFR-TKIs), has been developed for targeted therapy.

In the context of the present disclosure, the term “resistant to a treatment” means that the cell (e.g., tumor cell) exhibits reduced, diminished, or no responsiveness to a particular therapeutic agent (such as an EGFR-TKI), as compared with the same cell at an earlier time point (in the case of acquired resistance or adaptive resistance) or as compared with other cells of the same type (known as natural resistance) that respond to said therapeutic agent. As to the acquired resistance, the resistance to a therapeutic agent develops in a cell over time, typically during the course of treatment with said therapeutic agent. In contrast, natural resistance occurs in cells that are predisposed to resistance to a therapeutic agent.

Throughout the present disclosure and appended claims, the terms “treatment” and “treating” are used to include preventative (e.g., prophylactic), curative, or palliative treatment that results in a desired pharmaceutical and/or physiological effect. Preferably, the effect is therapeutic in terms of partially or completely curing or preventing tumor genesis, progression, or metastasis. Also, the terms “treatment” and “treating” as used herein refer to the application or administration of the present pharmaceutical composition to a subject, who has been to diagnosed with epidermoid carcinoma (such as non-small cell lung cancer) or exhibits a symptom of or predisposition toward epidermoid carcinoma, with the purpose to partially or completely alleviate, ameliorate, relieve, delay onset of, inhibit progression of, reduce severity of, and/or reduce incidence of one or more symptoms or features of epidermoid carcinoma. In particular, the present treatment is useful in overcoming the tumor cell's resistance to tyrosine kinase inhibitors such as EGFR-TKIs. Generally, a “treatment” includes not just the improvement of symptoms or decrease of markers of the disease, but also a cessation or slowing of progress or worsening of a symptom that would be expected in absence of treatment. Beneficial or desired clinical results of the present treatment include, but are not limited to, alleviation of one or more symptom(s) of epidermoid carcinoma, diminishment of size and/or volume of epidermoid carcinoma, stabilized (i.e., not worsening) state of epidermoid carcinoma, or delay or slowing of progression, metastasis, or remission of epidermoid carcinoma, whether partial or total, detectable or undetectable.

As used herein, the terms “overcome resistance” and “enhance susceptibility” toward certain agent(s) are used interchangeably in the present disclosure, and are meant to encompass the phenomenon in which the level or amount or degree of resistance to a therapeutic agent (e.g., a TKI) in a previously-resistant cell is diminished, reduced or reversed such that the cell, after the treatment, exhibits a measurable degree of responsiveness (or increased responsiveness) or an enhanced susceptibility to the same therapeutic agent, or another therapeutic agent that targets the same signaling pathway, as compared to the cell in its resistant state.

As used herein, the term “cancer” refers to all types of cancer or malignant neoplasm or tumors found in animals, including leukemia, carcinoma, melanoma, and sarcoma. The term “epidermoid carcinoma,” also referred to as “squamous cell carcinoma,” includes a subset of carcinomas that affect cells of squamous epithelial origins. Specifically, epidermoid carcinoma occurs in/at skin, lips, mouth, esophagus, urinary bladder, prostate, lungs, vagina, and cervix, among others.

The term “effective amount” as used herein refers to the quantity of a component (e.g., gefitinib) which is sufficient to yield a desired response (such as an inhibitory effect and/or a therapeutic effect). As to the effective amount of a targeted therapy sensitizer, said effective amount is the quantity of the targeted therapy sensitizer that is sufficient to elicit a synergistic effect with gefitinib so that the efficacy of gefitinib for treating epidermoid carcinoma in a subject in need thereof is improved or enhanced. Effective amount may be expressed, for example, in grams, milligrams, or micrograms, or as milligrams per kilogram of body weight (mg/kg). The term also refers to an amount of a pharmaceutical composition containing an active component or combination of components. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the structure of the compounds or its derivatives.

As used herein, the term “synergism” or “synergistic,” when referring to the synergism between gefitinib and the targeted therapy sensitizer, means that the two interact in ways that enhance or magnify one or more therapeutic and/or inhibitory effects of those drugs. That is, the combination of the two causes a greater effect than simply the sum of the individual effects of each drug if they were used separately.

As used herein, a “pharmaceutically acceptable” component is one that is suitable for use with humans and/or animals without undue adverse side effects (such as toxicity, irritation, and allergic response) commensurate with a reasonable benefit/risk ratio. Also, each excipient must be “acceptable” in the sense of being compatible with the other ingredients of the pharmaceutical formulation. The excipient can be in the form of a solid, semi-solid, or liquid diluent, cream or a capsule.

The terms “subject” and “patient” are used interchangeably herein to refer to a mammal that is treatable with the present pharmaceutical composition and/or method. The term “mammal” refers to all members of the class Mammalia, including humans, primates, domestic and farm animals (such as rabbit, pig, sheep, and cattle), rodents (such as mouse, rat, guinea pig, marmots, hamster), and zoo, sports or pet animals. The term “subject” is intended to refer to both the male and female gender unless one gender is specifically indicated. In some embodiments, the subject is a human being with epidermoid carcinoma that is resistant to an EGFR-TKI inhibitor.

The present disclosure is based, at least, on the finding that the combined administration of gefitinib, rapamycin, and a substituted quinolone (e.g., chloroquine) synergistically increase the antitumor efficacy of the targeted therapy therapeutic, gefitinib. In particular, experiments (as provided below) conducted using the EGFR-TKI resistant A549 cells indicate that the combined administration of these three drugs overcomes the resistance to the EGFR tyrosine kinase inhibitors. Accordingly, the first aspect of the present disclosure is directed to a pharmaceutical composition comprising gefitinib and a targeted therapy sensitizer that comprises rapamycin and a substituted quinolone.

According to various embodiments of the present disclosure, the pharmaceutical composition comprises an effective amount of gefitinib and the targeted therapy sensitizer in an amount sufficient to synergistically improve or enhance the efficacy of the gefitinib for treating epidermoid carcinoma in the subject. The pharmaceutical composition also comprises a pharmaceutically acceptable excipient.

The substituted quinolines useful in the invention may be prepared by synthetic techniques that are well known in the art and there have been many commercially-available substituted quinolines. In certain embodiments, suitable substituted quinolines include 4-aminoquinoline, 8-aminoquinoline, and hydroxymethylquinoline, hydroxychloroquine, chloroquine, amodiaquine, amopyroquine, bis-pyroquine, cycloquine, oxychloroquine, sontoquine, amoproquine, primaquine, mefloquine, quinacrine, tebuquine, quinine, thalidomide, sulfasalazine, and sulfapyridine, or pharmaceutically acceptable salt or enantiomer or prodrug thereof. A particularly example of preferred substituted quinolines used in the present examples is chloroquine or pharmaceutically acceptable salt thereof (e.g., chloroquine phosphate).

According to some embodiments of the present disclosure, the rapamycin and the substituted quinoline are present in the targeted therapy sensitizer in a weight ratio of about 10:1 to 1:5,000; preferably, 5:1 to 1:4,000; more preferably, 2:1 to 1:3,000. For example, the weight ratio between the rapamycin and the substituted quinoline is 10, 9.5, 9, 8.5, 8, 7.5, 7, 6.5, 6, 5.5, 5, 4.5, 4, 3.5, 3, 2.5, 2, 1.9, 1.8, 1.7, 1.6, 1.5, 1.4, 1.3, or 1.2:1, or 1:1, 1.25, 1.75, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 25, 30, 35, 40, 45, 50, 55, 60, 65, 70, 75, 80, 85, 90, 95,100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 950, 1,000, 1,100, 1,200, 1,300, 1,400, 1,500, 1,600, 1,700, 1,800, 1,900, 2,000, 2,500, 3,000, 3,500, 4,000, 4,500, or 5,000.

According to some embodiments of the present disclosure, the gefitinib and the targeted therapy sensitizer are present in the pharmaceutical composition in a weight ratio of about 10:1 to 1:100; preferably, 5:1 to 1:50; more preferably, 1:1 to 1:25. For example, the ratio between gefitinib and the targeted therapy sensitizer is about 10, 9.5, 9, 8.5, 8, 7.5, 7, 6.5, 6, 5.5, 5, 4.5, 4, 3.5, 3, 2.9, 2.8, 2.7, 2.6, 2.5, 2.4, 2.3, 2.2, 2.1, 2, 1.9, 1.8, 1.7, 1.6, 1.5, 1.4, 1.3, 1.2, or 1.1:1, or 1:1, 1.5, 2, 2.5, 3, 3.5, 4, 4.2, 4.5, 5, 5.5, 6, 6.5, 7, 7.5, 8, 8.5, 9, 9.5, 10, 11, 12, 13, 14, 15, 16, 17, 18, 19, 20, 25, 30, 35, 40, 45, 50, 55, 60, 65, 70, 75, 80, 85, 90, 95, or 100.

According to various embodiments of the present disclosure, the epidermoid carcinoma may be lung cancer, colon cancer or breast cancer. In certain embodiments, the epidermoid carcinoma is non-small cell lung cancer. In some cases, the non-small cell lung cancer is resistant to at least one tyrosine kinase inhibitor. Examples of tyrosine kinase inhibitors include, but are not limited to, gefitinib and erlotinib.

According to various embodiments of the present disclosure, the pharmaceutical composition is prepared in accordance with acceptable pharmaceutical procedures, such as described in Remington: The Science and Practice of Pharmacy, 20^{th} edition, ed. Alfonoso R. Gennaro, Lippincott Williams & Wilkins (2000).

The choice of a pharmaceutically acceptable excipient to be used in conjunction with the components of the present composition is basically determined by the way the pharmaceutical composition is to be administered. Further, according to various embodiments of the present disclosure, all of the three components (including, gefitinib, rapamycin, and the substituted quinoline) may be formulated in a single excipient that can be provided in a single dose unit. Alternatively, each component is formulated in one excipient; in this case, the three components may be provided in three separate dose units, or two or three of the components are then mixed together or formulated with an additional excipient to obtain one dose unit. For example, the rapamycin and the substituted quinoline may be formulated into one dose unit, while the gefitinib alone is formulated into another dose unit.

The pharmaceutical composition according to the present disclosure or components thereof may be administered by any suitable route, for example, by oral or parenteral (such as, intravenous, subcutaneous, intramuscular, intrathecal, intraperitoneal, intrarectal, viginal, nasal, intragastric, intratracheal, pulmonary, intratumoral or peritumoral injection) administration. Alternatively, the pharmaceutical composition or components thereof may be administered via an implant. As could be appreciated, in the case where the three components are provided in at least two dose units, these dose units may be administered via the same or different routes. Still optionally, these dose units may be given simultaneously or sequentially in time.

A pharmaceutical composition suitable for oral administration may be any orally acceptable dosage form including capsules, tablets, emulsions and aqueous suspensions, dispersions, and solutions. For example, the present pharmaceutical composition or component(s) thereof may be formulated into tablets containing various excipients such as microcrystalline cellulose, sodium citrate, calcium carbonate, dicalcium phosphate, and glycine; along with various disintegrants such as starch, alginic acid and certain silicates; together with granulation binders like polyvinylpyrrolidone, sucrose, gelatin, and acacia. Tablets can additionally be prepared with enteric coatings. Additionally, lubricating agents such as magnesium stearate, sodium lauryl sulfate, and talc may be added in the tablet form. For oral administration in a capsule form, solid fillers (such as, dried corn starch, milk sugar, and high molecular weight polyethylene glycols) may be employed in gelatin capsules. When aqueous dosage forms are desired for oral administration, the present pharmaceutical composition or component(s) thereof may be suspended or dissolved in a suitable solvent, optionally combined with emulsifying or suspending agents. If desired, certain sweetening, flavoring, or coloring agents, dyes, or diluents can be added.

Regarding parenteral administration, the present pharmaceutical composition or component(s) thereof may be formulated with a pharmaceutically acceptable excipient such as a sterile aqueous solution, which is preferably isotonic with the body fluid of the recipient. Such formulations may be prepared by dissolving or suspending the solid active ingredient in water containing physiologically compatible substances such as sodium chloride, glycine and the like, and having a buffered pH compatible with physiological conditions to produce an aqueous solution, and rendering said solution sterile. Other diluents or solvent suitable for manufacturing sterile injectable solution or suspension include, but are not limited to, 1,3-butanediol, mannitol, water, and Ringer's solution. Fatty acids, such as oleic acid and its glyceride derivatives are also useful for preparing injectables, as are natural pharmaceutically-acceptable oils, such as olive oil or castor oil. These oil solutions or suspensions may also contain alcohol diluent or carboxymethyl cellulose or similar dispersing agents. Other commonly used surfactants such as Tweens or Spans or other similar emulsifying agents or bioavailability enhancers that are commonly used in manufacturing pharmaceutically acceptable dosage forms can also be used for the purpose of formulation.

Still optionally, pharmaceutical compositions of the present invention can also comprise various pharmaceutically-acceptable additives well known to the art. Said additives include, but are not limited to, drying agent, anti-itch agents, anti-foaming agents, buffers, neutralizing agents, pH adjusting agents, coloring agents, discoloring agents, emollients, emulsifying agents, emulsion stabilizers, viscosity builders, humectants, odorants, preservatives, antioxidants, chemical stabilizers, thickening agents, stiffening agents, or suspending agents.

In another aspect, the present disclosure is directed to a method for treating epidermoid carcinoma in a subject in need of by administering to the subject a pharmaceutical composition according to any of the above-mentioned aspect/embodiments of the present disclosure. As could be appreciated, the above description regarding the species, amounts, formulations, administration routes of the drug components in the pharmaceutical composition according to embodiments of the present disclosure are also applicable in the method discussed below.

In particular, embodiments of the present disclosure provide a method for enhancing the susceptibility of a subject having epidermoid carcinoma toward a tyrosine kinase inhibitor. Specifically, the method comprises the steps of administering to the subject, an effective amount of the targeted therapy sensitizer and an effective amount of gefitinib, such that the targeted therapy sensitizer enhances the susceptibility (or responsiveness) of the epidermoid carcinoma toward gefitinib.

According to various embodiments of the present disclosure, the epidermoid carcinoma may be lung cancer, colon cancer, or breast cancer. In certain embodiments, the present pharmaceutical composition is useful in treating non-small cell lung cancer (NSCLC); in particular, advanced NSCLC such as those in subjects resistant to at least one tyrosine kinase inhibitor, such as gefitinib or erlotinib.

As discussed above, the administration of the present pharmaceutical composition may be carried out in any suitable routes, and the components of the present pharmaceutical composition may be administered via the same or different routes, simultaneously or sequentially in time. According to some preferred embodiments of the present disclosure, the tyrosine kinase inhibitor is administered in the same or different regimen of that of the targeted therapy sensitizer, meaning the tyrosine kinase inhibitor and the targeted therapy sensitizer may be administered at the same or different dose and/or frequency during the treatment course.

For example, in the case where the targeted therapy sensitizer is formulated in one dose unit, while the gefitinib is formulated in another dose unit, the targeted therapy sensitizer may be administered prior to, concurrently with, or after the administration of the gefitinib. When the two dose units are not administered concurrently, the time interval between the administrations of said two dose units is 12 hours at most. For example, the time interval nay be 1 2, 3, 4, 5, 10, 15, 20, or 25 minutes, or 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, or 12 hours. Preferably, the time interval may be less than 6 hours; more preferably, less than 5 hours.

Further, the targeted therapy sensitizer and gefitinib can be administered at the same or different frequencies. For example, during one course of the treatment, the targeted therapy sensitizer may be given first followed by multiple doses of gefitinib. Alternatively, after the administration of the first dose of the targeted therapy sensitizer, a second dose of the targeted therapy sensitizer is given after one or multiple doses of gefitinib. Still alternatively, at the beginning of one treatment course, two doses of targeted therapy sensitizer may be administered to the subject, in which multiple doses of gefitinib are administered in between. As could be appreciated by persons having ordinary skill in the art, the present invention is not limited to the above-mentioned dosage regimens, which are provided for illustrative purpose only.

The above-mentioned dosage regimes are also applicable in case where the three components are formulated in three different dosage units. For example, the dosage units of substituted quinoline and rapamycin are administered at substantially the same time and the same frequency, while the dosage unit of gefitinib is administered at the same time or different time, with the same or a different frequency.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

Materials and Methods

1. Materials

Rapamycin and chloroquine (C6628 Chloroquine diphosphate) were purchased from Sigma-Aldrich (St. Louis, Mo., USA). Gefitinib was purchased from Cayman Chemical (Michigan, USA).

2. Cell Culture

Erlotinib-resistant human NSCLC cell line, NCI-H1975 (obtained from Dr. Chih-Hsin Yang, Department of Oncology, National Taiwan University Hospital, Taipei, Taiwan), was cultured in RPMI-1640 medium (Gibco-BRL) supplemented with 10% fetal bovine serum (Gibco-BRL). Gefitinib-resistant NSCLC cell line, A549 (No. 60074), was obtained from Bioresource Collection and Research Center (BCRC), Hsinchu, Taiwan, and cultured in DMEM (Gibco-BRL) medium supplemented with 10% fetal bovine serum.

3. Cell Apoptosis Assay

After incubation with drugs as described in the Examples below, cells on the plate were washed with PBS and dissociated with trypsin/EDTA. The apoptosis assay was performed using the Annexin V (BD Pharmingen) apoptosis detection kit per the manufacturer's instruction. Briefly, the collected cells were washed with PBS for three times. The cells were treated with 222.5 μL of binding buffer and dyed with 10 μL of propidium iodide (PI) and 2.5 μL Annexin V-FITC, then the cells were incubated at 2-8° C. in the dark for move the reaction to an environment with low temperature and no light for 10 minutes. Cell apoptosis was then analyzed by FACSCalibur flow cytometer (Becton Dickinson Bioscience, Mountain View, Calif.) and the percentage of apoptotic cells (or the apoptotic ratio; AR) was calculated using the CellQuest software. Each experiment was performed in triplicate, and the results were expressed as mean±SD.

4. Determination of Acridine Orange (AO) Accumulation in Acidic Vesicular Organelles (AVO5)

After incubation with drugs as described in the Examples below, cells were removed from plate with trypsin/EDTA. Cells were than stained with acridine orange (A6014; from Sigma-Aldrich) at a final concentration of 1 g/ml for a period of 15 minutes. Green (510-530 nm) and red (about 650 nm) fluorescence emission from 10⁴ cells illuminated with blue (488 nm) excitation light was measured with a FACSCalibur using CellQuest software.

5. Cell Viability Assay and the Calculation of IC₅₀

The cells were plated at 5×10⁴/ml in 100 μL of culture medium in a 96-well microtiter plate, followed by incubation with drugs. Cell viability after the treatment was determined using 3-(4,5-dimethylthiazol-2-yl)-5(3-carboxymethoxy-phenyl)-2-(4-sulfophenyl)-2H-tetrazolium (MTS) assay (Promega, Madison, Wis.) per the manufacturer's instruction. Absorbance was measured at 490 nm for MTS assay with an enzyme-linked immunosorbent assay plate reader from MTX Lab Systems, Inc. (Vienna, Va.). The 50% inhibitory concentration (IC₅₀) was calculated using CalcuSyn (Biosoft, Ferguson) software. Each experiment was performed in triplicate, and the results were expressed as mean±SD.

6. Calculation of Combination Index

Combination experiments were performed using the fixed IC₅₀ ratio method. Combination Index (CI) values were calculated using Biosoft CalcuSyn program (Ferguson, Mo.). The dose-effect relationship analyzed using the median-effect equation was first obtained for each single drug by its serial dilution. Constant ratios of three drugs were used to obtain the isobologram ED values. The CI value for each combination treatment was then calculated using the same software.

7. Immunoblotting

Protein lysates obtained from treated cells were quantified using a colorimetric detection assay (BCA Protein Assay, Pierce, Rockford, Ill., USA). Equal amounts of protein lysates (100 μg) were separated by gel electrophoresis on 7% or 15% sodium dodecyl sulfate-polyacrylamide gels, and transferred to Immobilon-P membranes (Millipore Corporation, Bedford, Mass., USA). Blots were probed by anti-LC3 (Novus), anti-Beclin1, anti-BCL-2, anti-caspase 3, anti-PARP, anti-mTOR, anti-phospho-mTOR, anti-phospho-STAT3, anti-ERK1/2 and anti-phospho-ERK1/2 (Cell signaling), and anti-GAPDH (Sigma). The corresponding peroxidase-conjugated secondary antibody (anti-mouse IgG-horseradish peroxidase; Dako) was detected using ECL Western blot reagents (Millipore).

### Example 1

### Targeted Therapy Sensitizer Plus Gefitinib Synergistically Increase Percentage of Apoptotic A431 Cells

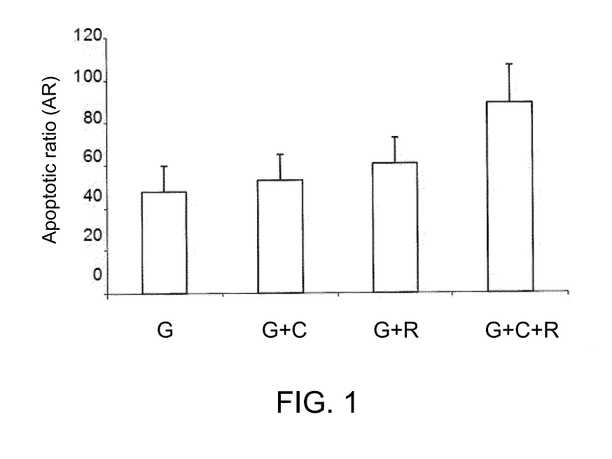

A431 cells were inoculated in a 6-wells plate at the density of 1×10⁶ cells/well. Solution of 5 μM gefitinib, 5 μM gefitinib and 10 μM chloroquine, 5 μM gefitinib and 10 nM rapamycin, or 5 μM gefitinib, 10 μM chloroquine and 10 nM rapamycin was then added into each well at room temperature. Thereafter, the plate was placed in a 37° C. incubator and cultured for 48 hours, before the cells were subjected to apoptosis assay. Results were summarized in FIG. 1.

As illustrated in FIG. 1, gefitinib alone resulted in about 50% apoptotic cell death in gefitinib-resistant A431 cells (group G), and the co-administration of gefitinib and chloroquine (G+C) or gefitinib and rapamycin (G+R) slightly increased the percentage of apoptotic cells to about 52% or 60%. On the other hand, while gefitinib was administered in conjunction with both chloroquine and rapamycin, the percentage of apoptotic gefitinib-resistant A431 cells increased drastically to about to 90%. These preliminary data suggested that while the chloroquine or rapamycin alone failed to increase the percentage of apoptotic gefitinib-resistant cells to gefitinib, yet the combined use of chloroquine and rapamycin as a targeted therapy sensitizer did increase the susceptibility of gefitinib-resistant cells toward gefitinib, with the observance of a synergistic increase in the apoptotic cell death in gefitinib-resistant cells.

### Example 2

### Targeted Therapy Sensitizer Plus Gefitinib Synergistically Increase Percentage of Apoptotic H1975 Cells

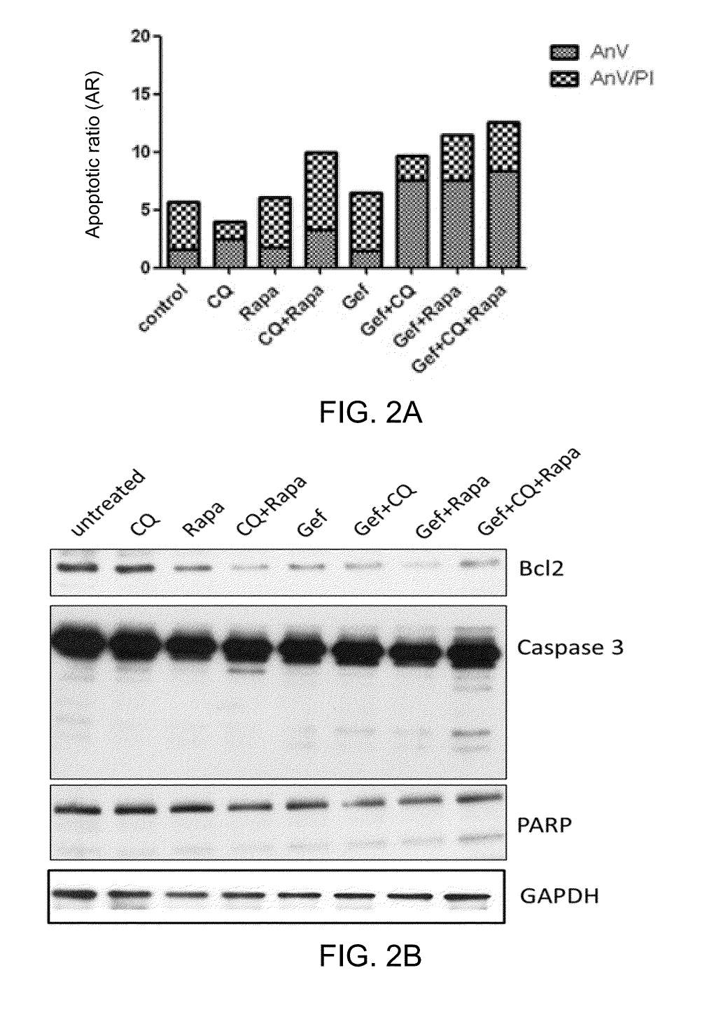

H1975 cells were treated with various drugs or combinations thereof (37.5 μM chloroquine, 7.5 μM rapamycin and 5 μM gefitinib) for 72 hours. The percentage of apoptotic cells of various treatment groups were summarized in FIG. 2A.

Referring to FIG. 2A, the combined administration of two drug components induced higher level of apoptosis in erlotinib-resistant H1975 cells; in particular, the percentage of apoptotic cells is 10% for chloroquine plus rapamycin (CQ+Rapa), 9.7% for gefitinib plus chloroquine (Gef+CQ), and 11.5% for gefitinib plus rapamycin (Gef+Rapa). On the other hand, the combined use of gefitinib plus chloroquine and rapamycin resulted in a 12.6% of apoptotic death in H1975 cells, suggesting the existence of a synergistic effect when the three components drug combination was employed.

Immunoblotting was performed to investigate the presence of three apoptotic markers (caspase 3, PARP, and Bcl2) in the treated cells, and the results were provided in FIG. 2B.

As was evident from FIG. 2B, the treatment with gefitinib plus chloroquine and rapamycin substantially induced the cleavage of caspase 3, as compared with the administration of one or two of the above-mentioned drugs. PARP was also cleaved, after the administration of gefitinib plus chloroquine and rapamycin. As to Bcl2, its expression in H1975 cells was substantially inhibited after the treatment with gefitinib plus chloroquine and rapamycin, as compared with those treated with one- or two-component composition.

### Example 3

### Targeted Therapy Sensitizer Plus Gefitinib Synergistically Inhibit Cell Proliferation of H1975 and A549 Cells

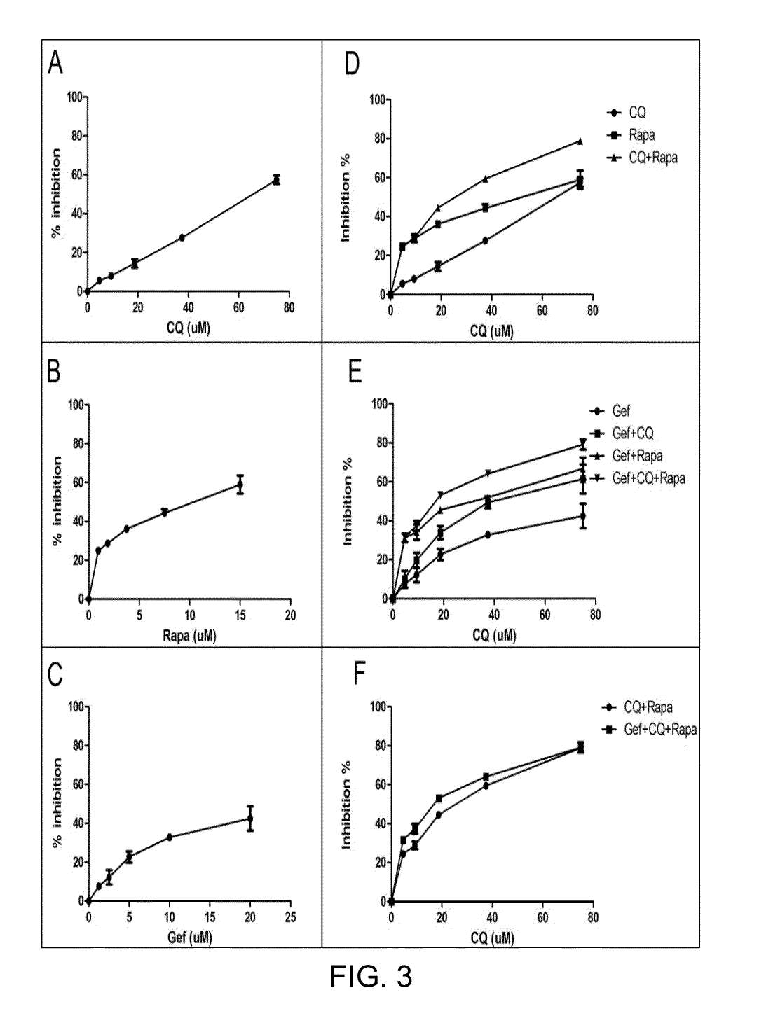

To determine the effect of gefitinib, chloroquine, or rapamycin, or a combination thereof on the cell viability of EGFR-TKI resistant cells, erlotinib-resistant H1975 cells or A549 cells were treated with serially-diluted drug-containing solutions for 72 hours and then subjected to the MTS assay. Results of the inhibition of cell proliferation regarding H1975 cells were provided in FIG. 3, and the combination index values of each combination treatment given at various effective dose equivalents (including ED₃₀, ED₅₀, ED₇₅, and ED₉₀) regarding H1975 cells and A549 cells were respectively summarized in Table 1 and Table 2.

Calculated IC₅₀ values for chloroquine (panel A, FIG. 3), rapamycin (panel B, FIG. 3), and gefitinib (panel C, FIG. 3) were 75 μM and 20 μM, respectively. Accordingly, in the following combined treatment, chloroquine, rapamycin, and gefitinib were given at a molar ratio of 15:3:4 (weight ratio of about 16:9:6). For example, in panels D-F of FIG. 3, when the concentration of chloroquine was 60 μM, the concentrations of rapamycin and gefitinib were 12 μM and 16 μM, respectively.

Referring to FIG. 3, the growth of H1975 cells was further inhibited by the combined treatment of chloroquine and rapamycin as compared with the treatment of chloroquine or rapamycin alone (panel D). Similarly, the growth of H1975 cells was also inhibited by the combined use of chloroquine, rapamycin, and the gefitinib, as compared with the treatment of gefitinib alone, gefitinib plus chloroquine, or gefitinib plus rapamycin (panel E), or chloroquine plus rapamycin (panel F).

TABLE 1

| | Dose | | | |
|---|---|---|---|---|
| H1975 Cell | ED₃₀ | ED₅₀ | ED₇₅ | ED₉₀ |
| C + R | 1.11 | 0.73 | 0.55 | 0.55 |
| G + C | 0.87 | 0.87 | 0.90 | 0.96 |
| G + R | 0.71 | 0.80 | 1.04 | 1.55 |
| G + C + R | 0.77 | 0.88 | 1.06 | 1.28 |

The synergistic effect of each treatment was compared using combination index (CI), in which CI of 0.9 to 1.1 indicated synergistic effects. The data in Table 1 indicated that the combination of gefitinib, chloroquine, and rapamycin synergistically inhibited the proliferation of the erlotinib-resistant H1975 cells when the composition was given at the ED₇₀ dose that was effective in inhibiting up to 75% cell proliferation.

Regarding the gefitinib-resistant A549 cells, the data in Table 2 demonstrated that when administered at the ED₃₀ or ED₅₀ dose, the combination of gefitinib, chloroquine, and rapamycin synergistically inhibit the proliferation of the A549 cells.

TABLE 2

| | Dose | | | |
|---|---|---|---|---|
| A549 Cell | ED₃₀ | ED₅₀ | ED₇₅ | ED₉₀ |
| C + R | 0.77 | 0.58 | 0.83 | 1.55 |
| G + C | 0.88 | 1.25 | 1.49 | 1.89 |
| G + R | 1.19 | 1.49 | 3.74 | 12.53 |
| G + C + R | 0.94 | 1.03 | 1.16 | 1.31 |

The experimental data provided hereinabove confirmed that the co-administration of gefitinib and a targeted therapy sensitizer, which consists of rapamycin and a substituted quinoline (e.g., chloroquine), helps the EGFR-TKI resistant cells (such as, gefitinib- or erlotinib-resistant cells) to overcome the EGFR-TKI resistance at least by inducing tumor cell apoptosis and inhibiting tumor cell proliferation. In other words, the responsiveness of EGFR-TKI resistant cells to gefitinib (an EGFR tyrosine kinase inhibitor) is increased in a measurable degree under the sensitizing action of rapamycin and chloroquine, as compared with that of the cells in their drug-resistant state.

It will be understood that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method for enhancing the susceptibility of a subject having epidermoid carcinoma toward a tyrosine kinase inhibitor, comprising,
administering to the subject an effective amount of a targeted therapy sensitizer consisting of rapamycin and a substituted quinoline; and
administering to the subject an effective amount of gefitinib.

2. The method of claim 1, wherein the substituted quinoline is chloroquine or a pharmaceutically acceptable salt thereof.

3. The method of claim 2, wherein the rapamycin and the substituted quinoline are present in the targeted therapy sensitizer in a weight ratio of 10:1 to 1:5,000.

4. The method of claim 3, wherein the rapamycin and the substituted quinoline are present in the targeted therapy sensitizer in a weight ratio of 5:1 to 1:4,000.

5. The method of claim 4, wherein the rapamycin and the substituted quinolone are present in the targeted therapy sensitizer in a weight ratio of 2:1 to 1:3,000.

6. The method of claim 1, wherein the gefitinib and the targeted therapy sensitizer are administered in a weight ratio of 10:1 to 1:100.

7. The method of claim 6, wherein the gefitinib and the targeted therapy sensitizer are administered in a weight ratio of 5:1 to 1:50.

8. The method of claim 7, wherein the gefitinib and the targeted therapy sensitizer are administered in a weight ratio of 1:1 to 1:25.

9. The method of claim 1, wherein the epidermoid carcinoma is non-small cell lung cancer.

10. The method of claim 9, wherein the non-small cell lung cancer is resistant to at least one tyrosine kinase inhibitor.

11. The method of claim 10, wherein the tyrosine kinase inhibitor is gefitinib or erlotinib.

12. The method of claim 1, wherein the tyrosine kinase inhibitor is gefitinib or erlotinib.

13. The method of claim 1, wherein the targeted therapy sensitizer and the gefitinib are respectively administered in single dose or multiple doses.

14. The method of claim 13, wherein the targeted therapy sensitizer is administered at a regimen same as or different from that of gefitinib.

15. The method of claim 14, wherein the targeted therapy sensitizer is administered prior to the administration of gefitinib.

16. The method of claim 14, wherein the targeted therapy sensitizer is administered concurrently with the administration of gefitinib.

17. The method of claim 14, wherein the targeted therapy sensitizer is administered after the administration of gefitinib.

18. The method of claim 14, wherein,
the targeted therapy sensitizer is administered to the subject in at least two doses; and
one or more doses of gefitinib are administered between any two doses of the targeted therapy sensitizer.

19. The method of claim 1, wherein the substituted quinoline is chloroquine phosphate, and the weight ratio between rapamycin and chloroquine phosphate and is 1:1 to 1:2.

20. The method of claim 1, wherein the subject is a human being.
